(19) United States
(12) Patent Application Publication
(10) Pub. No.: US 2026/0204415 A1
(43) Pub. Date: Jul. 16, 2026

(54) METHOD, COMPUTING DEVICE, AND RECORDING MEDIUM FOR EARLY PREDICTING SEPTIC SHOCK THROUGH BIO-DATA ANALYSIS BASED ON ARTIFICIAL INTELLIGENCE

(71) Applicant: Spass Inc., Seoul (KR)

(72) Inventors: Yonghwan KIM, Seoul (KR); San LEE, London (CA)

(73) Assignee: Spass Inc., Seoul (KR)

(21) Appl. No.: 19/558,888

(22) Filed: Mar. 6, 2026

Related U.S. Application Data
(63) Continuation of application No. PCT/KR2024/013601, filed on Sep. 9, 2024.

(30) Foreign Application Priority Data
Sep. 11, 2023 (KR) 10-2023-0120549

(51) Int. Cl. G16H 50/20; A61B 5/00; G06N 3/096; G16H 10/40; G16H 15/00

(52) U.S. Cl. CPC G16H 50/20; A61B 5/412; A61B 5/7267; A61B 5/7275; G06N 3/096; G16H 10/40; G16H 15/00

(57) ABSTRACT

Provided are a method, device, and recording medium of early predicting septic shock through bio-data analysis based on artificial intelligence. A method of early predicting septic shock through bio-data analysis based on artificial intelligence, which is performed by a computing device, according to various embodiments of the present invention includes collecting time series-based bio-data measured in real time from a user and predicting an occurrence of septic shock in the user by analyzing the collected bio-data based on an artificial intelligence model, in which the predicting of the occurrence of the septic shock includes analyzing bio-data at a future time point predicted based on the collected bio-data to early predict the occurrence of the septic shock at the future time point, when sepsis is detected in the user by analyzing the collected bio-data.

### CROSS-REFERENCE TO RELATED APPLICATIONS

This present application is a Continuation of International Application No. PCT/KR 2024/013601 filed on Sep. 9, 2024, which is based upon and claims the benefit of priority to Korean Patent Application No. 10-2023-0120549, filed on Sep. 11, 2023, disclosures of which are incorporated herein by reference in its entirety.

### BACKGROUND

### 1. Technical Field

Various embodiments of the present invention relate to a method, device, and recording medium for early predicting septic shock through bio-data analysis based on artificial intelligence.

### 2. Related Art

Sepsis refers to a total physical phenomenon that occurs when bacteria or bacterial toxins are present in the blood.

When two or more of fever symptoms where body temperature rises above 38° C. or where body temperature falls below 36° C. (hypothermia), a symptom (tachypnea) where a respiratory rate increases to more than 24 breaths per minute, a symptom (tachycardia) where a heart rate increases to more than 90 beats per minute, and a symptom where the number of white blood cells increases or significantly decreases in a blood test appear, it is called systemic inflammatory response syndrome (SIRS). When this systemic inflammatory response syndrome is caused by microbial infection, it is called sepsis.

When bacteria or toxins produced by bacteria appear in the blood, our body responds with various defense mechanisms. During the response process, patients show the above symptoms.

Vital organs may be damaged due to actions of various inflammatory mediators generated by a body's response to sepsis and the accompanying blood flow disorders. Sepsis accompanied by damage to vital organs is called severe sepsis, and when blood pressure is not maintained with general fluid therapy, it is called septic shock. Mortality naturally increases more in the severe sepsis than in sepsis, and increases more in septic shock than in severe sepsis.

When sepsis is not treated early, the patient's body response may escalate quite quickly, which may result in the patient dying within a few days. The progression of sepsis may be alleviated by early use of appropriate antibacterial agents and appropriate fluid therapy. Therefore, early treatment is essential to improve a patient's survival rate. It is difficult for medical staff to take medical action within 2 hours or within 24 hours at the latest after the appearance of early symptoms. Once sepsis has already progressed, it is difficult to improve the survival rate even with various devices and intensive treatment by specialized doctors.

Vital signs and blood tests are required to diagnose sepsis. The vital signs should be measured regularly by medical staff, and the blood tests require infrastructure capable of collecting and analyzing blood. Therefore, there was a problem in that it is difficult to monitor whether the sepsis occurs in real time in hospitals. In particular, sepsis is a major burden in areas with a low sociodemographic index. This is because these areas have insufficient medical resources for sepsis diagnosis and treatment.

### SUMMARY

The present invention provides a method, device, and recording medium for early predicting septic shock through bio-data analysis based on artificial intelligence that can diagnose and predict sepsis and septic shock in real time and more easily diagnose and predict sepsis and septic shock not only with professional measuring devices but also with sensor data collected through wearable devices such as smart watches, by analyzing time series-based bio-data (vital signs) collected in real time from users to determine whether sepsis or septic shock has occurred in the users.

The present invention provides a method, device, and recording medium for early predicting septic shock through bio-data analysis based on artificial intelligence that, when sepsis is detected by analyzing bio-data collected from users in real time, by predicting future bio-data based on current bio-data and early predicting the occurrence of septic shock by analyzing the future bio-data to detect the occurrence of septic shock, can dramatically reduce a mortality rate of sepsis patients.

Objects of the present invention are not limited to the above-mentioned objects. That is, other objects that are not mentioned may be obviously understood by those skilled in the art from the following description.

According to an embodiment of the present invention, a method of early predicting septic shock through bio-data analysis based on artificial intelligence, which is performed by a computing device, includes collecting time series-based bio-data measured in real time from a user and predicting an occurrence of septic shock in the user by analyzing the collected bio-data based on an artificial intelligence model, in which the predicting of the occurrence of the septic shock may include analyzing bio-data at a future time point predicted based on the collected bio-data to early predict the occurrence of the septic shock at the future time point, when sepsis is detected in the user by analyzing the collected bio-data.

The early prediction of the occurrence of septic shock at the future time point may include analyzing bio-data collected from the user at a first time point to determine whether sepsis occurs in the user at the first time point, predicting bio-data at a second time point after a predetermined time has elapsed from the first time point based on the bio-data collected at the first time point, when it is determined that the sepsis occurs, and analyzing the predicted bio-data at the second time point to determine whether the septic shock occurs in the user at the second time point.

The determination of whether the sepsis occurs in the user at the first time point may include determining whether the sepsis occurs in the user at the first time point by analyzing the bio-data collected at the first time point using a first artificial intelligence model, and the first artificial intelligence model may be a machine learning or deep learning-based classifier trained using a plurality of pieces of bio-data labeled with information on the occurrence of the sepsis as training data.

The predicting of the bio-data at the second time point may include predicting the bio-data at the second time point by analyzing the bio-data collected at the first time point using a second artificial intelligence model, and the second artificial intelligence model may be a deep learning-based forecaster trained using a plurality of pieces of bio-data collected from sepsis patients as learning data.

The determining of whether the sepsis occurs in the user at the second time point may include determining whether the sepsis occurs in the user at the second time point by analyzing the predicted bio-data at the second time point using a third artificial intelligence model, and the third artificial intelligence model may be a machine learning or deep learning-based classifier trained using a plurality of pieces of bio-data labeled with information on the occurrence of the septic shock as training data.

The training data may include the plurality of pieces of bio-data labeled with the occurrence of septic shock at a location corresponding to the time point at which the septic shock occurs, and the time point at which the septic shock occurs is an administration time point of a vasopressor.

The artificial intelligence model may include a plurality of artificial intelligence models that classify bio-data collected from a plurality of measuring devices with different attributes for each measuring device and are generated by being trained using each piece of the bio-data classified for each measuring device as training data, the plurality of artificial intelligence models may be models that are fine-tuned independently of each other based on weights corresponding to each of the plurality of measuring devices, and the weights corresponding to each of the plurality of measuring devices may be determined based on accuracy of sensor data measured through each of the plurality of measuring devices.

The collected bio-data may include a plurality of pieces of bio-data of different types, and the predicting of the occurrence of the septic shock may further include selecting at least one piece of the plurality of pieces of bio-data based on a type of measuring device that measures the plurality of pieces of bio-data, and predicting the occurrence of the septic shock in the user by analyzing only the selected at least one piece of bio-data through the artificial intelligence model.

The collected bio-data may include a plurality of pieces of bio-data of different types, and the predicting of the occurrence of the septic shock may further include extracting a plurality of result data by individually analyzing the plurality of pieces of bio-data through the artificial intelligence model, predicting the occurrence of the septic shock in the user using the plurality of extracted result data, assigning a weight to each of the plurality of extracted result data based on the type of measuring device that measures the plurality of pieces of bio-data, and predicting the occurrence of the septic shock in the user using the plurality of weighted result data.

The artificial intelligence model may include a plurality of artificial intelligence models that classify a plurality of pieces of bio-data collected in different environments for each situation and are generated by being trained using each piece of the plurality of pieces of bio-data classified for each situation as training data, and the predicting of the occurrence of septic shock may further include, when bio-data in a specific situation is collected from the user, predicting the occurrence of the septic shock in the user by adopting an artificial intelligence model trained using the bio-data collected in the specific situation as the training data among the plurality of artificial intelligence models to analyze the bio-data collected in the specific situation.

The artificial intelligence model may include an explainable artificial intelligence (XAI) model and a generative artificial intelligence model, and the method may further include deriving basis data for a result of early prediction of the occurrence of the septic shock at the future time point using the XAI model, deriving response data for the result of early prediction of the occurrence of the septic shock at the future time point using the generative artificial intelligence model, and generating a result report including the derived basis data and the derived response data and providing the generated result report, as the result of early prediction of the occurrence of septic shock at the future time point.

A computing device for performing a method of early predicting septic shock through bio-data analysis based on artificial intelligence includes a processor, a network interface, a memory, and a computer program loaded into the memory and executed by the processor, in which the computer program may include an instruction to collect time series-based bio-data measured in real time from a user, and an instruction to predict an occurrence of septic shock in the user by analyzing the collected bio-data based on an artificial intelligence model, and the instruction to predict the occurrence of the septic shock may include an instruction to analyze the bio-data at a future time point predicted based on the collected bio-data to early predict the occurrence of the septic shock at the future time point, when sepsis is detected in the user by analyzing the collected bio-data.

According to another embodiment of the present invention, a computing device-readable recording medium, which is coupled to a computing device and on which a computer program for executing a method of early predicting septic shock through bio-data analysis based on artificial intelligence is recorded, wherein the method may include collecting time series-based bio-data measured in real time from a user, and predicting an occurrence of septic shock in the user by analyzing the collected bio-data based on an artificial intelligence model, and the predicting of the occurrence of the septic shock may include analyzing the bio-data at a future time point predicted based on the collected bio-data to early predict the occurrence of the septic shock at the future time point, when sepsis is detected in the user by analyzing the collected bio-data.

Other specific details of the invention are included in the detailed description and drawings.

### BRIEF DESCRIPTION OF DRAWINGS

FIG. 1 is a diagram illustrating a system for early predicting septic shock through bio-data analysis based on artificial intelligence according to an embodiment of the present invention.

FIG. 2 is a diagram illustrating a hardware configuration of a computing device for performing a method of early predicting septic shock through bio-data analysis based on artificial intelligence according to another embodiment of the present invention.

FIG. 3 is a flowchart of a method of early predicting septic shock through bio-data analysis based on artificial intelligence according to another embodiment of the present invention.

FIG. 4 is a diagram exemplarily illustrating bio-data applicable to various embodiments.

FIG. 5 is a flowchart for describing the method for early predicting occurrence of septic shock based on an artificial intelligence model in various embodiments.

FIGS. 6 and 7 are diagrams illustrating a process of deriving result data by analyzing bio-data through an artificial intelligence model in various embodiments.

### DESCRIPTION OF EXAMPLE EMBODIMENTS

Various advantages and features of the present invention and methods accomplishing them will become apparent from the following description of embodiments with reference to the accompanying drawings. However, the present invention is not limited to embodiments to be described below, but may be implemented in various different forms, these embodiments will be provided only in order to make the present invention complete and allow those skilled in the art to completely recognize the scope of the present invention, and the present invention will be defined by the scope of the claims.

Terms used in the present specification are for explaining embodiments rather than limiting the present invention. Unless otherwise stated, a singular form includes a plural form in the present specification. Throughout this specification, the terms “comprise” and/or “comprising” will be understood to imply the inclusion of stated constituents but not the exclusion of any other constituents. Like reference numerals refer to like components throughout the specification and “and/or” includes each of the components mentioned and includes all combinations thereof. Although the terms “first,” “second,” and the like are used to describe various components, it goes without saying that these components are not limited by these terms. These terms are used only to distinguish one component from other components. Therefore, it goes without saying that the first component mentioned below may be the second component in the technical scope of the present invention.

Unless defined otherwise, all terms (including technical and scientific terms) used in the present specification have the same meanings commonly understood by those skilled in the art to which the present invention pertains. In addition, terms defined in commonly used dictionary are not ideally or excessively interpreted unless explicitly defined otherwise.

Further, the term “unit” or “module” used herein means a software component or a hardware component such as a field programmable gate array (FPGA) or an application-specific integrated circuit (ASIC) and performs predetermined functions. However, the term “unit” or “module” is not meant to be limited to software or hardware. The “unit” or “module” may be stored in a storage medium that can be addressed or may be configured to regenerate one or more processors. Accordingly, for example, the “unit” or “module” includes components such as software components, object-oriented software components, class components, and task components, processors, functions, attributes, procedures, subroutines, segments of a program code, drivers, firmware, a microcode, a circuit, data, a database, data structures, tables, arrays, and variables. Functions provided in components, “units,” or “modules” may be combined into fewer components, “units,” or “modules” or further separated into additional components, “units,” or “modules.”

Spatially relative terms “below,” “beneath,” “lower,” “above,” “upper,” and the like may be used to easily describe the correlation between one component and other components as illustrated in drawings. The spatially relative terms should be understood as terms including different directions of components during use or operation in addition to the directions illustrated in the drawings. For example, when components illustrated in the drawings are turned up, a component described as “below” or “beneath” another component may be placed “above” the another component. Therefore, the exemplary term “below” can include both downward and upward directions. The components can also be aligned in different directions, and therefore the spatially relative terms can be interpreted according to the alignment.

In this specification, a computer means all kinds of hardware devices including at least one processor, and can be understood as including a software configuration which is operated in the corresponding hardware device according to the embodiment. For example, the computer may be understood as a meaning including all of smart phones, tablet PCs, desktops, laptops, and user clients and applications running on each device, but is not limited thereto.

Hereinafter, embodiments of the present invention will be described in detail with reference to the accompanying drawings.

Each operation described in this specification is described as being performed by the computer, but subjects of each operation are not limited thereto, and according to embodiments, at least some of each operations can also be performed on different devices.

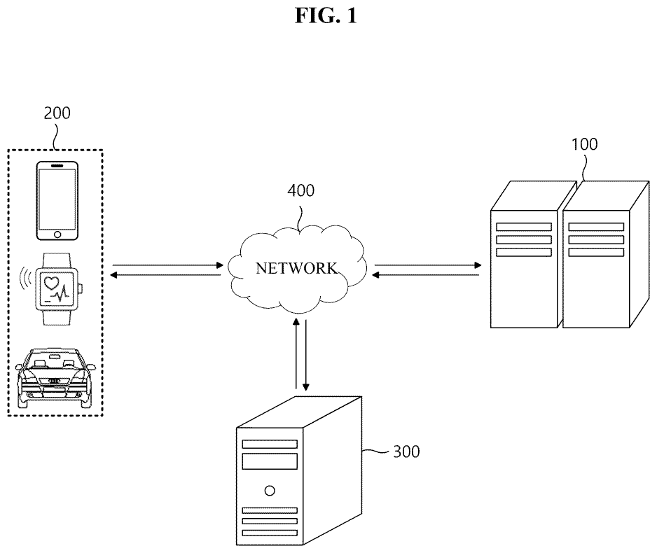

FIG. 1 is a diagram illustrating a system for early predicting septic shock through bio-data analysis based on artificial intelligence according to an embodiment of the present invention.

Referring to FIG. 1, the system for early predicting septic shock through bio-data analysis based on artificial intelligence according to an embodiment of the present invention includes a computing device 100, a user terminal 200, an external server 300, and a network 400.

Here, the system for early predicting septic shock through bio-data analysis based on artificial intelligence illustrated in FIG. 1 is according to an embodiment, and its components are not limited to the embodiment illustrated in FIG. 1, and may be added, changed, or deleted, if necessary.

In an embodiment, the computing device 100 may perform a process of early predicting septic shock through artificial intelligence-based bio-data analysis.

In various embodiments, the computing device 100 may collect time series-based bio-data measured in real time from users and analyze the bio-data through an artificial intelligence model to predict the occurrence of septic shock in the users.

Here, the artificial intelligence model may be a model trained using the bio-data as training data, and may be a model that predicts sepsis and septic shock as result data using the bio-data as input data.

The artificial intelligence model (e.g., a neural network) is composed of one or more network functions, and one or more network functions may be composed of a set of interconnected computational units, which may generally be referred to as “node.” These “nodes” may also be referred to as “neurons.” One or more network functions include at least one or more nodes. Nodes (or neurons) that constitute one or more network functions may be interconnected by one or more “links.”

Within the artificial intelligence model, one or more nodes connected through the link may form a relative relationship between the input node and output node. The concepts of the input node and the output node are relative, and any node in the relationship of the output node with respect to one node may be in the input node relationship in the relationship with another node, and vice versa. As described above, the relationship between the input node and the output node may be generated around the link. One or more output nodes may be connected to one input node through the link, and vice versa.

In the relationship between the input node and the output node connected through one link, a value of the output node may be determined based on data input to the input node. Here, the node connecting between the input node and the output node may have weights. The weights may be variable, and may vary by a user or algorithm in order for the artificial intelligence model to perform the desired functions. For example, when one or more input nodes are connected to one output node by the respective links, the value of the output node may be determined based on the values input to the input nodes connected to the output node and the weights set on the links corresponding to the respective input nodes.

As described above, the artificial intelligence model interconnects one or more nodes through one or more links to form the relationship between the input node and the output node within the artificial intelligence model. The characteristics of the artificial intelligence model may be determined according to the number of nodes and links within the artificial intelligence model, the correlation between nodes and links, and the weight values assigned to each link. For example, if there are two artificial intelligence models with the same number of nodes and links and different weight values between the links, the two artificial intelligence models may be recognized as different from each other.

Some of the nodes constituting the artificial intelligence model may constitute one layer based on distances from an initial input node. For example, a set of nodes with a distance n from the initial input node may constitute n layers. The distance from the initial input node may be defined by the minimum number of links that should be passed to reach the corresponding node from the initial input node. However, this definition of the layer is arbitrary for explanation purposes, and the order of the layer within the artificial intelligence model may be defined in a different way than described above. For example, the layer of the nodes may be defined by a distance from a final output node.

The initial input nodes may refer to one or more nodes, to which data is directly input without going through links in relationships with other nodes, among the nodes in the artificial intelligence model. Alternatively, the initial input nodes may refer to nodes that do not have other input nodes connected by the link in the relationship between the nodes based on the link within the artificial intelligence model network. Similarly, the final output nodes may refer to one or more nodes that do not have the output node in the relationship with other nodes among the nodes in the artificial intelligence model. In addition, hidden nodes may refer to nodes that constitute the artificial intelligence model rather than the first input node and the last output node. The artificial intelligence model according to the embodiment of the present invention may have more nodes of the input layer than the nodes of the hidden layer close to the output layer, and may be the artificial intelligence model in which the number of nodes decreases as it progresses from the input layer to the hidden layer.

The artificial intelligence model may include one or more hidden layers. The hidden node of the hidden layer may use an output of a previous layer and an output of surrounding hidden nodes as an input. The number of hidden nodes for each hidden layer may be the same or different. The number of nodes of the input layer may be determined based on the number of data fields of the input data and may be the same as or different from the number of hidden nodes. The input data input to the input layer may be calculated by the hidden node of the hidden layer and output by a fully connected layer (FCL) which is the output layer.

In various embodiments, the artificial intelligence model may be a deep learning model.

The deep learning model (e.g., a deep neural network (DNN)) may refer to a neural network including a plurality of hidden layers in addition to an input layer and an output layer. It is possible to identify latent structures of data by using the deep neural network. That is, it is possible to identify the latent structures (e.g., what objects are in the photo, what the content and emotion of the text are, what the content and emotion of the audio are, etc.) of a photo, text, video, sound, or music.

The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siamese network, and the like, but is not limited thereto.

In various embodiments, services for early prediction and notifying of septic shock through a process of early predicting septic shock through bio-data analysis based on artificial intelligence provided by the computing device 100 may be implemented in the form of an application, and users may run applications through devices (e.g., smart phones, wearable devices, etc.) capable of running the applications to use the services for early prediction and notifying of septic shock.

In various embodiments, the computing device 100 may be connected to the user terminal 200 through the network 400 and may provide various types of information derived by collecting various types of information (e.g., user information, user bio-data, etc.) required to perform a method of early predicting septic shock through bio-data analysis based on artificial intelligence from the user terminal 200 or provide various types of information (e.g., result report) derived by performing the method of early predicting septic shock through bio-data analysis based on artificial intelligence.

Here, the user terminal 200 may refer to any type of entity(s) in the system that has a mechanism for communication with the computing device 100. For example, the user terminal 200 may include a personal computer (PC), a notebook, a mobile terminal, a smart phone, a tablet personal computer (tablet PC), a wearable device, etc., and may include all types of terminals that may access wired/wireless networks. In addition, the user terminal 200 may include any computing device implemented by at least one of an agent, an application programming interface (API), and a plug-in. In addition, the user terminal 200 may include an application source and/or a client application. In addition, the user terminal 200 may include, but is not limited to, an infotainment system installed in a vehicle.

In addition, here, the network 400 may be a connection structure capable of exchanging information between respective nodes such as a plurality of terminals and servers. For example, the network 400 may include a local area network (LAN), a wide area network (WAN), the Internet (World Wide Web (WWW)), a wired/wireless data communication network, a telephone network, a wired/wireless television communication network, a controller area network (CAN), Ethernet, or the like.

Examples of the wireless data communication network may include 3G, 4G, 5G, 3^{rd} Generation Partnership Project (3GPP), 5^{th} GPP (5GPP), long term evolution (LTE), world interoperability for microwave access (WiMAX), Wi-Fi, Internet, a LAN, a wireless LAN (WLAN), a WAN, a personal area network (PAN), radio frequency, a Bluetooth network, a near-field communication (NFC) network, a satellite broadcast network, an analog broadcast network, a digital multimedia broadcasting (DMB) network, and the like, but are not limited thereto.

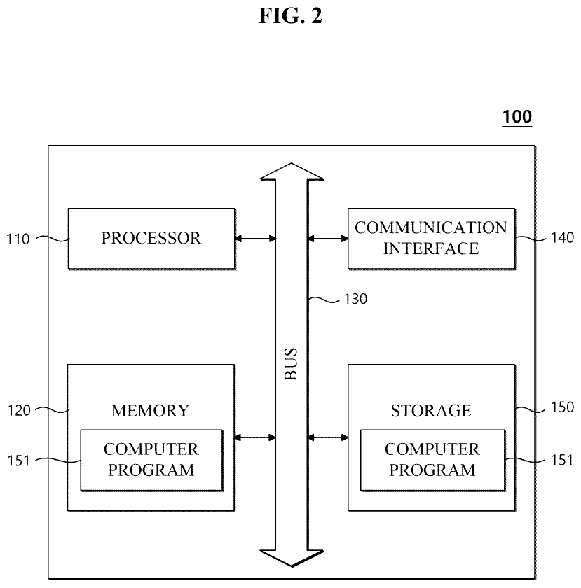

In an embodiment, the external server 300 may be connected to the computing device 100 through the network 400, and the computing device 100 may store and manage various types of information and data necessary to perform the method of early predicting septic shock through bio-data analysis based on artificial intelligence, or collect, store, and manage various types of information and data derived by performing the method of early predicting septic shock through bio-data analysis based on artificial intelligence. For example, the external server 300 may be a storage server separately provided outside the computing device 100, but is not limited thereto. Hereinafter, the hardware configuration of the computing device 100 that performs the method of early predicting septic shock through bio-data analysis based on artificial intelligence will be described with reference to FIG. 2.

FIG. 2 is a diagram illustrating a hardware configuration of a computing device for performing a method of early predicting septic shock through bio-data analysis based on artificial intelligence according to another embodiment of the present invention.

Referring to FIG. 2, according to various embodiments, the computing device 100 may include one or more processors 110, a memory 120 into which a computer program 151 executed by the processor 110 is loaded, a bus 130, a communication interface 140, and a storage 150 for storing the computer program 151. Here, only the components related to the embodiment of the present invention are illustrated in FIG. 2. Accordingly, those skilled in the art to which the present invention pertains may understand that other general-purpose components other than those illustrated in FIG. 2 may be further included.

The processor 110 controls an overall operation of each component of the computing device 100. The processor 110 may include a central processing unit (CPU), a micro processor unit (MPU), a micro controller unit (MCU), a graphics processing unit (GPU), or any type of processor well known in the art of the present invention.

In addition, the processor 110 may perform an operation on at least one application or program for executing the method according to the embodiments of the present invention, and the computing device 100 may include one or more processors.

In various embodiments, the processor 110 may further include a random access memory (RAM) (not illustrated) and a read-only memory (ROM) for temporarily and/or permanently storing signals (or data) processed in the processor 110. In addition, the processor 110 may be implemented in the form of a system-on-chip (SoC) including at least one of a graphics processing unit, a RAM, and a ROM.

The memory 120 stores various types of data, commands and/or information. The memory 120 may load the computer program 151 from the storage 150 to execute methods/operations according to various embodiments of the present invention. When the computer program 151 is loaded into the memory 120, the processor 110 may perform the method/operation by executing one or more instructions constituting the computer program 151. The memory 120 may be implemented as a volatile memory such as a RAM, but the technical scope of the present invention is not limited thereto.

The bus 130 provides a communication function between the components of the computing device 100. The bus 130 may be implemented as various types of buses, such as an address bus, a data bus, and a control bus.

The communication interface 140 supports wired/wireless Internet communication of the computing device 100. In addition, the communication interface 140 may support various communication methods other than the Internet communication. To this end, the communication interface 140 may include a communication module well known in the art of the present invention. In some embodiments, the communication interface 140 may be omitted.

The storage 150 may non-temporarily store the computer program 151. When the computing device 100 performs the process of early predicting septic shock through bio-data analysis based on artificial intelligence, the storage 150 may store various types of information necessary to provide the process of early predicting septic shock through bio-data analysis based on artificial intelligence.

The storage 150 may include a nonvolatile memory, such as a ROM, an erasable programmable ROM (EPROM), an electrically EPROM (EEPROM), and a flash memory, a hard disk, a removable disk, or any well-known computer-readable recording medium in the art to which the present invention pertains.

The computer program 151 may include one or more instructions to cause the processor 110 to perform methods/operations according to various embodiments of the present invention when loaded into the memory 120. That is, the processor 110 may perform the method/operation according to various embodiments of the present invention by executing the one or more instructions.

In an embodiment, the computer program 151 may include one or more instructions to perform the method of early predicting septic shock through bio-data analysis based on artificial intelligence that includes collecting time series-based bio-data measured in real time from a user and predicting the occurrence of septic shock in the user by analyzing the collected bio-data based on an artificial intelligence model.

Operations of the method or algorithm described with reference to the embodiment of the present invention may be directly implemented in hardware, in software modules executed by hardware, or in a combination thereof. The software module may reside in a RAM, a ROM, an EPROM, an EEPROM, a flash memory, a hard disk, a removable disk, a compact disc read-only memory (CD-ROM), or in any form of computer-readable recording medium known in the art to which the invention pertains.

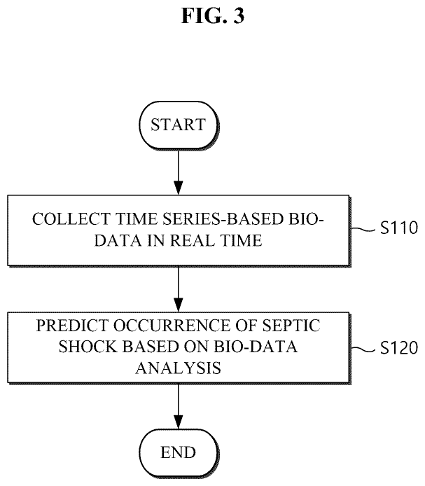

The components of the present invention may be embodied as a program (or application) and stored in a medium for execution in combination with a computer which is hardware. The components of the present invention may be executed in software programming or software elements, and similarly, embodiments may be realized in a programming or scripting language such as C, C++, Java, and assembler, including various algorithms implemented in a combination of data structures, processes, routines, or other programming constructions. Functional aspects may be implemented in algorithms executed on one or more processors. Hereinafter, the method of early predicting septic shock through bio-data analysis based on artificial intelligence performed by the computing device 100 will be described with reference to FIG. 3.

FIG. 3 is a flowchart of a method of early predicting septic shock through bio-data analysis based on artificial intelligence according to another embodiment of the present invention.

Referring to FIG. 3, in operation S110, the computing device 100 may collect the bio-data measured from the user.

In various embodiments, the computing device 100 may be connected to a measuring device through the network 400 and may collect the user's bio-data measured through the measuring device.

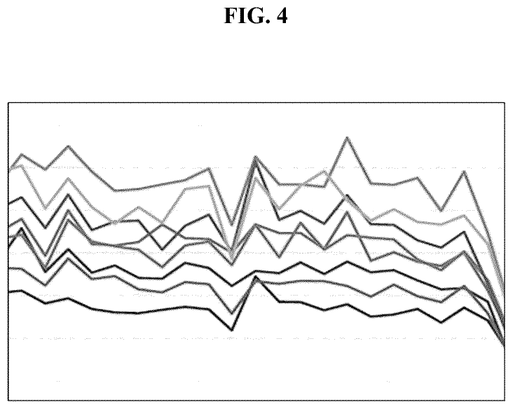

Here, the user's bio-data may be time series-based bio-data (e.g., FIG. 4) measured in real time from the user through the measuring device. For example, the user's bio-data may include a shock index (SI), respiration (Rr), saturation of percutaneous oxygen (SpO2), temperature (Temp), and heart rate (Hr), and mean arterial pressure (MAP) of the user, but is not limited thereto, and various bio-data that may determine the conditions of the user may be applied.

In addition, here, the measuring device that measures the user's bio-data may be a medical device including a plurality of sensors for measuring different types of bio-data or a plurality of medical devices each including a plurality of sensors, but is not limited thereto, and the measuring device may be applied to any component that may measure the user's bio-data, such as the user terminal 200 (e.g., user's mobile, wearable device, or infotainment system of a vehicle in which a user rides).

In various embodiments, the computing device 100 may directly receive the user's bio-data from medical staff in charge of the user who wishes to predict the occurrence of the septic shock. However, it be applied to various methods of collecting user's bio-data without being thereto.

In various embodiments, the computing device 100 may collect the user's bio-data at preset intervals (e.g., 1 hour). Here, the preset cycle may be determined based on the occurrence history of sepsis and/or septic shock of the user, but is not limited thereto.

In operation S120, the computing device 100 may predict the occurrence of septic shock of the user by analyzing the bio-data collected through operation S110.

In general, the septic shock is caused by a rapid drop in blood pressure during the occurrence and progression of sepsis. That is, since the sepsis occurs and progresses before septic shock occurs, the computing device 100 may preferentially predict whether sepsis occurs by

analyzing the user's bio-data by taking this into account, and may predict whether septic shock occurs when it is determined that the sepsis has occurred.

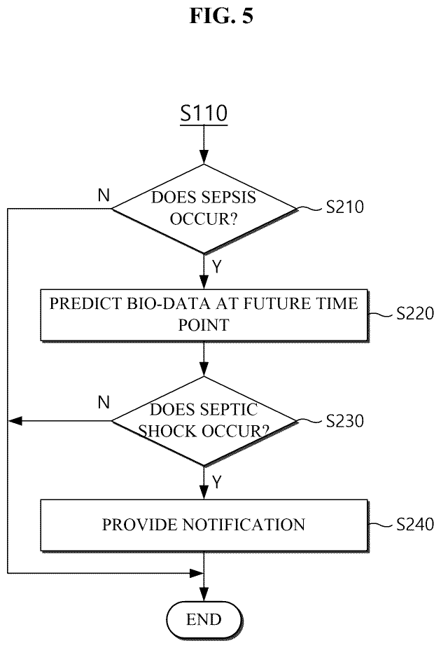

In various embodiments, the computing device 100 may detect the occurrence of sepsis in the user by analyzing the bio-data at the current time point through a pre-trained artificial intelligence model, and may early predict the occurrence of septic shock at one future time point by analyzing bio-data of a future time point predicted based on bio-data at a current time point when the occurrence of sepsis in the user is detected. Hereinafter, the method for early prediction of septic shock performed through an artificial intelligence model will be described in more detail with reference to FIGS. 5 to 7.

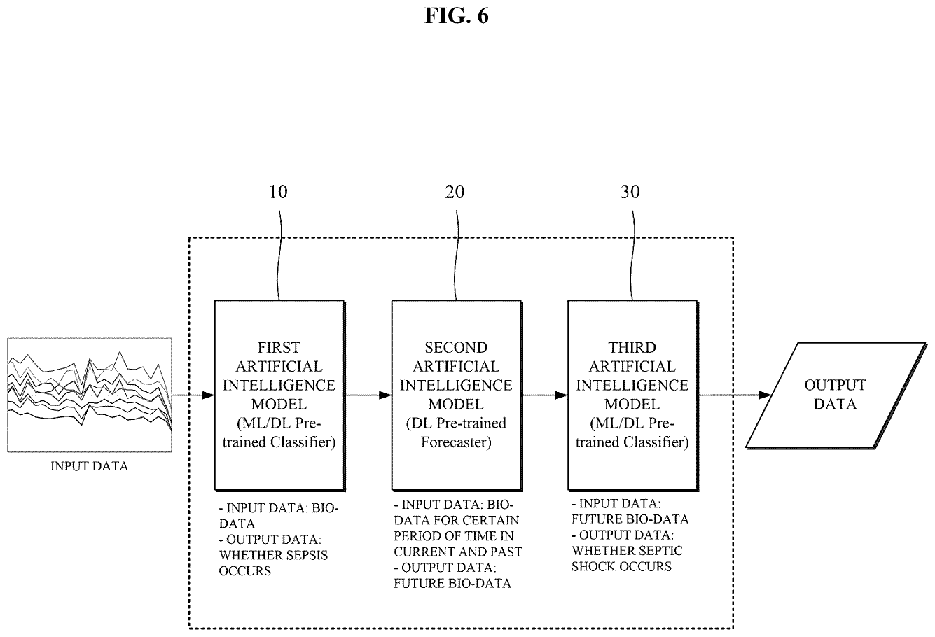

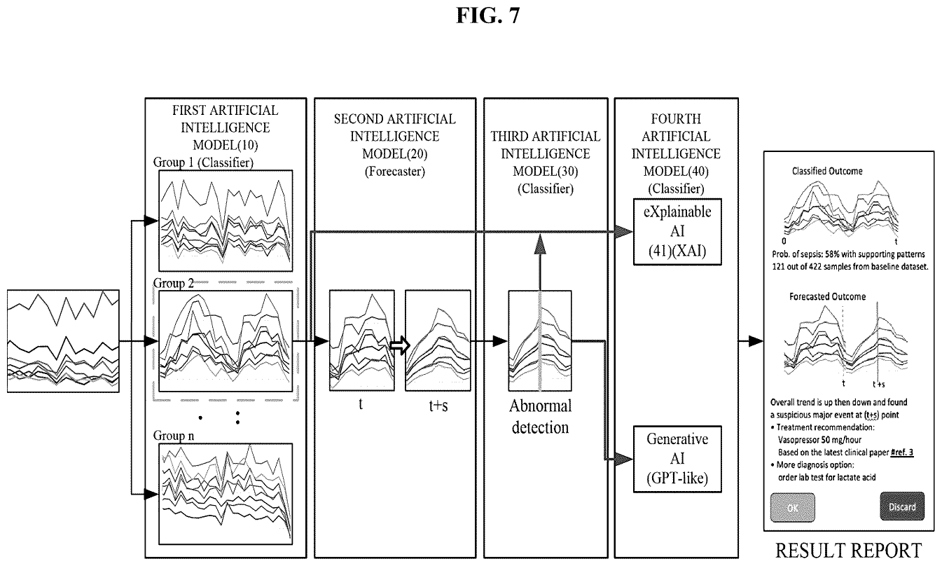

FIG. 5 is a flowchart for describing a method for early predicting occurrence of septic shock based on an artificial intelligence model in various embodiments, and FIGS. 6 and 7 are diagrams illustrating the process of deriving result data by analyzing bio-data through an artificial intelligence model in various embodiments.

Referring to FIGS. 5 to 7, in operation S210, when the bio-data is collected from the user at a first time point (e.g., current time point), the computing device 100 may determine whether sepsis has occurred at the first time point by analyzing the bio-data collected at the first time point.

In various embodiments, the computing device 100 may determine whether sepsis has occurred at the first time point by analyzing the bio-data collected at the first time point using the first artificial intelligence model 10.

Here, the first artificial intelligence model 10 is a model trained using a plurality of pieces of bio-data labeled with information on the occurrence of sepsis as training data, and may be a model that uses specific bio-data as input data and outputs whether sepsis has occurred as result data.

In addition, here, the first artificial intelligence model 10 may be a machine learning or deep learning-based classifier, and may be a model that analyzes the bio-data to classify a user into any one of a plurality of predefined groups (e.g., normal group, mild sepsis group, and severe sepsis group), but is not limited thereto.

In various embodiments, when a plurality of different types of sensor data are collected from a user, the computing device 100 may extract a plurality of result data by individually analyzing each of the plurality of sensor data through the first artificial intelligence model 10 and determine whether the sepsis has occurred at the first time point by combining the plurality of extracted result data. For example, the computing device 100 may acquire six result data by analyzing each of the SI, Rr, SpO2, Temp, Hr, and MAP collected from the user through the first artificial intelligence model 10 and may determine whether the sepsis has finally occurred by collecting six results (e.g., hard voting or soft voting). However, the present invention is not limited thereto.

In operation S220, when it is determined that sepsis has occurred through operation S210, the computing device 100 may predict bio-data at a second time point.

Here, the second time point may mean a future time point in which a predetermined time has elapsed from the first time point which is the current time point. For example, the second time point may be a time point that has passed 3 to 4 hours based on the current time point, but is not limited thereto.

In various embodiments, the computing device 100 may predict the bio-data at the second time point by analyzing the bio-data collected at the first time point using the second artificial intelligence model 20.

Here, the second artificial intelligence model 20 is a model trained using a plurality of pieces of bio-data collected from sepsis patients as training data, and may be a model that uses bio-data at a specific time point as input data and outputs bio-data at a time point after the predetermined time has elapsed from the specific time point.

In addition, the second artificial intelligence model 20 may be a deep learning-based forecaster (Forecaster), but is not limited thereto.

In various embodiments, when a plurality of different types of sensor data are collected from a user, the computing device 100 may predict the plurality of sensor data at the second time point corresponding to each of the plurality of sensor data at the first time point by individually analyzing each of the plurality of sensor data through the second artificial intelligence model 20. However, the present invention is not limited thereto.

Meanwhile, when it is determined that sepsis has not occurred through operation S210, the computing device 100 may terminate the operation of the process of early predicting septic shock through bio-data analysis based on artificial intelligence.

In operation S230, the computing device 100 may determine whether septic shock has occurred in the user at the second time point by analyzing the bio-data predicted through operation S220, that is, the bio-data at the second time point.

In various embodiments, the computing device 100 may determine whether the septic shock has occurred in the user at the second time point by analyzing the bio-data at the second time point using the third artificial intelligence model 30.

Here, the third artificial intelligence model 30 is a model trained using a plurality of pieces of bio-data labeled with information on the occurrence of septic shock as training data, and may be a model that uses specific bio-data as input data and outputs whether the septic shock has occurred as result data. For example, the third artificial intelligence model 30 may be a model trained using as training data a plurality of pieces of bio-data labeled with the occurrence of septic shock at a location corresponding to the time point at which the septic shock has occurred.

Here, the bio-data used as the training data for the third artificial intelligence model 30 requires the labeling operation at the time point at which the septic shock has occurred. Since the time point at which septic shock has occurred is a somewhat ambiguous time standard, there is a problem in that it is difficult to accurately specify the corresponding time point and the performance of the third artificial intelligence model 30 trained using such training data may deteriorate.

Typically, since the septic shock is caused by a rapid drop in blood pressure during the progress of sepsis, by setting the time point at which septic shock has occurred is set as an administration time point of the vasopressor when septic shock occurs in consideration of that it is essential to immediately administer the vasopressor, it is possible to construct a more accurate training data set and thus, construct a more reliable third artificial intelligence model 30.

In addition, here, the third artificial intelligence model 30 may be a machine learning or deep learning-based classifier, and may be a model that analyzes the bio-data to classify a user into any one of a plurality of predefined groups (e.g., normal group, caution group, and risk group), but is not limited thereto.

In various embodiments, when a plurality of sensor data corresponding to each of a plurality of different types of sensor data is predicted through the second artificial intelligence model 20, the computing device 100 may extract a plurality of result data by individually analyzing each of the plurality of sensor data through the third artificial intelligence model 30 and determine

whether the septic shock has occurred at the second time point by combining a plurality of extracted result data. For example, the computing device 100 may acquire six result data by analyzing each of the SI, Rr, SpO2, Temp, Hr, and MAP predicted through the second artificial intelligence model 20 and may determine whether the septic shock has finally occurred by collecting six results (e.g., hard voting or soft voting). However, the present invention is not limited thereto.

In various embodiments, the computing device 100 may select an artificial intelligence model corresponding to a measuring device that measures bio-data from a user among a plurality of pre-constructed artificial intelligence models, and determine whether septic shock has occurred in the user by analyzing the selected artificial intelligence model to analyze the user's bio-data.

Typically, in the case of professional medical devices that measure bio-data, relatively reliable sensor data may be obtained, whereas in the case of wearable devices, relatively less reliable sensor data compared to the medical devices may be collected due to various factors such as the performance of the wearable device, the condition, situation, and posture of the user, etc.

In addition, even when it is the same type of measuring device, the accuracy and reliability of each sensor are different depending on the attributes of the measuring device (e.g., in the case of the wearable device, the version, brand, etc., of the wearable device).

In addition, in some cases, there are measuring devices that can restrictively collect only some of the bio-data required in the process of determining whether sepsis and septic shock have occurred.

Considering this, the computing device 100 may individually prepare a plurality of artificial intelligence models corresponding to each of a plurality of measuring devices having different attributes in advance and adopt the artificial intelligence model corresponding to the measuring device that collects the bio-data from the user, thereby determining whether the sepsis and septic shock have occurred in the user.

More specifically, first, the computing device 100 may classify the bio-data collected from the plurality of measuring devices with different attributes for each measuring device, and train different models using each bio-data classified for each measuring device as training data, thereby constructing a plurality of artificial intelligence models corresponding to each of the plurality of measuring devices in advance.

In this case, the plurality of artificial intelligence models may be models that are fine-tuned independently of each other based on weights corresponding to each of the plurality of measuring devices.

Here, the weights corresponding to each of the plurality of measuring devices are determined based on the accuracy of sensor data measured through each of the plurality of measuring devices. For example, the computing device 100 may assign a higher weight to bio-data with relatively high accuracy among different types of bio-data compared to bio-data with relatively low accuracy, but is not limited thereto.

Thereafter, when the bio-data is collected through a specific measuring device, the computing device 100 adopts an artificial intelligence model corresponding to a specific measuring device among the plurality of artificial intelligence models and analyzes the bio-data to determine whether the sepsis and septic shock have occurred in the user, thereby deriving more accurate results.

In various embodiments, when a plurality of different types of bio-data are obtained from a user, the computing device 100 may analyze the plurality of pieces of bio-data through the artificial intelligence model to determine whether sepsis has occurred in the user and whether the septic shock has occurred in the user but may select at least one piece of the plurality of pieces of bio-data based on the type of measuring device that measures the plurality of pieces of bio-data, and analyze only the at least one bio-data selected through the artificial intelligence model, thereby predicting the occurrence of septic shock in the user.

For example, the computing device 100 may define in advance the type of bio-data with high accuracy for each measuring device based on the bio-data collection performance for each measuring device, and when the plurality of pieces of bio-data are collected from the specific measuring device, the computing device 100 may selectively analyze the type of bio-data preset for the specific measuring device, that is, the type of bio-data with high accuracy among the plurality of pieces of bio-data collected from the specific measuring device to determine whether the sepsis and septic shock have occurred in the user, thereby deriving more accurate results.

In various embodiments, when the plurality of different types of bio-data are acquired from the user, the computing device may analyze each of the plurality of pieces of bio-data through the artificial intelligence model and extract the plurality of result data, and collect the plurality of result data to determine whether sepsis and septic shock have occurred in the user, but may assign weights to each of the plurality of result data based on the type of measuring device that measures the plurality of pieces of bio-data and determine whether sepsis and septic shock have occurred in the user using a plurality of weighted result data. For example, the computing device 100 may assign greater weights to result data derived from bio-data with high accuracy compared to result data derived from bio-data with low accuracy based on the type of measuring device that measures the plurality of pieces of bio-data, there deriving more accurate results.

In various embodiments, the computing device 100 may select the artificial intelligence model corresponding to the measuring device that measures the user's situation at the time of measuring the bio-data from the user from the user among the plurality of pre-constructed artificial intelligence models, and determine whether the septic shock has occurred in the user by analyzing the selected artificial intelligence model to analyze the user's bio-data.

More specifically, first, the computing device 100 may classify the plurality of pieces of bio-data collected in different environments for each situation (e.g., while driving, during daily life, while exercising, during meditation (immobile posture), etc.), and train different models using each piece of the plurality of pieces of bio-data classified for each situation as training data, thereby constructing in advance the plurality of artificial intelligence models corresponding to each of the plurality of different situations.

In this case, the plurality of artificial intelligence models may be fine-tuned models independently of each other based on the weights corresponding to each of the plurality of different situations, and the weights here may be determined based on the accuracy of bio-data collection for each situation.

Thereafter, when the bio-data in the specific situation is collected from the user, the computing device 100 adopts an artificial intelligence model corresponding to the specific situation among the plurality of artificial intelligence models and analyzes the bio-data to determine whether the sepsis and septic shock have occurred in the user, thereby deriving more accurate results. For example, when the bio-data is collected while the user is driving, the user's bio-data may be analyzed through the artificial intelligence model trained using the bio-data collected while driving as the training data, thereby more accurately determining whether sepsis and septic shock have occurred in the driving user.

In various embodiments, when the computing device 100 predicts the occurrence of septic shock in the user as it performs the bio-data analysis on the user, the computing device 100 may provide the user with the notification corresponding to the occurrence of septic shock.

In this case, the attributes (e.g., size, type, cycle, frequency, range of provision, etc.) of the notification may be determined based on the predicted occurrence possibility of septic shock in the user.

For example, the computing device 100 may calculate the occurrence probability of septic shock in the user based on the result data derived by analyzing the bio-data through the artificial intelligence model and classify the user into a normal level, a caution level, or a risk level according to the calculated probability, may not provide a separate notification when the user is at the normal level, and provide a notification of a first size to the user every first cycle when the user is at the caution level, and provide a notification of a second size greater than the first size every second cycle shorter than the first cycle not only to the user but also to a user's pre-registered guardian (e.g., family member, medical professional, etc.).

As another example, the computing device 100 may not provide a separate notification when the user is classified into the normal level as the bio-data of the driving user is analyzed through the artificial intelligence model, provide the notification to the user's mobile when the user is classified into the caution level, provide notifications not only through a user's mobile and a vehicle's infotainment system, but also through mobile phones of passengers who boards the vehicle when the user is classified into the risk level.

In various embodiments, the computing device 100 may generate a result report corresponding to the derived result through the first artificial intelligence model 10, the second artificial intelligence model 20, and the third artificial intelligence model 30 through a fourth artificial intelligence model 40, and provide the generated result report to the user.

For example, as illustrated in FIG. 7, the fourth artificial intelligence model 40 may include an explainable artificial intelligence model (XAI) 41 and a generative artificial intelligence model 42, the computing device 100 may derive basis data for the results of early prediction of the occurrence of septic shock at one future time point using the explainable artificial intelligence model 41, derive response data for the results of early prediction of the occurrence of septic shock at one future time point using the generative artificial intelligence model 42, and generate the result report including the basis data and response data as a result of early prediction of the occurrence of septic shock at one future time point and provide the generated result report to the user.

The above-described method of early predicting septic shock through bio-data analysis based on artificial intelligence was described with reference to the flowchart illustrated in the drawing. For a simple description, the method of early predicting septic shock through bio-data analysis based on artificial intelligence has been described by showing a series of blocks, but the present invention is not limited to the order of the blocks, and some blocks may be performed in an order different from that shown and performed in the present specification, or may be performed concurrently. In addition, new blocks not described in the present specification and drawings may be added, or some blocks may be deleted or changed.

According to various embodiments of the present invention, it is possible to diagnose and predict sepsis and septic shock in real time and more easily diagnose and predict sepsis and septic shock not only with the professional measuring devices but also with the sensor data collected through the wearable devices such as smart watches, by analyzing the time series-based bio-data (vital signs) collected in real time from the users to determine whether sepsis or septic shock has occurred in the users.

In addition, when the sepsis is detected by analyzing the bio-data collected from the users in real time, by predicting the future bio-data based on the current bio-data and early predicting the occurrence of septic shock by analyzing the future bio-data to detect the occurrence of septic shock, it is possible to dramatically reduce the mortality rate of sepsis patients.

The effects of the present invention are not limited to the above-described effects, and other effects that are not mentioned may be obviously understood by those skilled in the art from the following description.

Although exemplary embodiments of the present invention has been described with reference to the accompanying drawings, those skilled in the art to which the present disclosure belongs will appreciate that various modifications and alterations may be made without departing from the spirit or essential feature of the present invention. Therefore, it is to be understood that embodiments described above are illustrative rather than being restrictive in all aspects.

What is claimed is:

1. A method of early predicting septic shock through bio-data analysis based on artificial intelligence, which is performed by a computing device, the method comprising:
collecting time series-based bio-data measured in real time from a user; and
predicting an occurrence of septic shock in the user by analyzing the collected bio-data based on an artificial intelligence model,
wherein the predicting of the occurrence of the septic shock includes analyzing bio-data at a future time point predicted based on the collected bio-data to early predict the occurrence of the septic shock at the future time point, when sepsis is detected in the user by analyzing the collected bio-data.

2. The method of claim 1, wherein the early prediction of the occurrence of the septic shock at the future time point includes:
analyzing bio-data collected from the user at a first time point to determine whether sepsis occurs in the user at the first time point;
predicting bio-data at a second time point after a predetermined time has elapsed from the first time point based on the bio-data collected at the first time point, when it is determined that the sepsis occurs; and
analyzing the predicted bio-data at the second time point to determine whether the septic shock occurs in the user at the second time point.

3. The method of claim 2, wherein the determination of whether the sepsis occurs in the user at the first time point includes:
determining whether the sepsis occurs in the user at the first time point by analyzing the bio-data collected at the first time point using a first artificial intelligence model, and
the first artificial intelligence model is a machine learning or deep learning-based classifier trained using a plurality of pieces of bio-data labeled with information on the occurrence of the sepsis as training data.

4. The method of claim 2, wherein the predicting of the bio-data at the second time point includes:
predicting the bio-data at the second time point by analyzing the bio-data collected at the first time point using a second artificial intelligence model, and
the second artificial intelligence model is a deep learning-based forecaster trained using a plurality of pieces of bio-data collected from sepsis patients as learning data.

5. The method of claim 2, wherein the determining of whether the sepsis occurs in the user at the second time point includes:
determining whether the sepsis occurs in the user at the second time point by analyzing the predicted bio-data at the second time point using a third artificial intelligence model, and
the third artificial intelligence model is a machine learning or deep learning-based classifier trained using a plurality of pieces of bio-data labeled with information on the occurrence of the septic shock as training data.

6. The method of claim 5, wherein the training data includes the plurality of pieces of bio-data labeled with the occurrence of the septic shock at a location corresponding to the time point at which the septic shock occurs, and the time point at which the septic shock occurs is an administration time point of a vasopressor.

7. The method of claim 1, wherein the artificial intelligence model includes a plurality of artificial intelligence models that classify bio-data collected from a plurality of measuring devices with different attributes for each measuring device and are generated by being trained using each piece of the bio-data classified for each measuring device as training data,
the plurality of artificial intelligence models are models that are fine-tuned independently of each other based on weights corresponding to each of the plurality of measuring devices, and
the weights corresponding to each of the plurality of measuring devices are determined based on accuracy of sensor data measured through each of the plurality of measuring devices.

8. The method of claim 1, wherein the collected bio-data includes a plurality of pieces of bio-data of different types, and
the predicting of the occurrence of the septic shock further includes selecting at least one piece of the plurality of pieces of bio-data based on a type of measuring device that measures the plurality of pieces of bio-data, and predicting the occurrence of the septic shock in the user by analyzing only the selected at least one piece of bio-data through the artificial intelligence model.

9. The method of claim 1, wherein the collected bio-data includes a plurality of pieces of bio-data of different types, and
the predicting of the occurrence of the septic shock further includes extracting a plurality of result data by individually analyzing the plurality of pieces of bio-data through the artificial intelligence model, predicting the occurrence of the septic shock in the user using the plurality of extracted result data, assigning a weight to each of the plurality of extracted result data based on the type of measuring device that measures the plurality of pieces of bio-data, and predicting the occurrence of the septic shock in the user using the plurality of weighted result data.

10. The method of claim 1, wherein the artificial intelligence model includes a plurality of artificial intelligence models that classify a plurality of pieces of bio-data collected in different environments for each situation and are generated by being trained using each piece of the plurality of pieces of bio-data classified for each situation as training data, and
the predicting of the occurrence of the septic shock further includes, when bio-data in a specific situation is collected from the user, predicting the occurrence of the septic shock in the user by adopting an artificial intelligence model trained using the bio-data collected in the specific situation as the training data among the plurality of artificial intelligence models to analyze the bio-data collected in the specific situation.

11. The method of claim 1, wherein the artificial intelligence model includes an explainable artificial intelligence (XAI) model and a generative artificial intelligence model,
the method further includes:
deriving basis data for a result of early prediction of the occurrence of the septic shock at the future time point using the XAI model;
deriving response data for the result of early prediction of the occurrence of the septic shock at the future time point using the generative artificial intelligence model; and
generating a result report including the derived basis data and the derived response data and providing the generated result report, as the result of early prediction of the occurrence of septic shock at the future time point.

12. A computing device for performing a method of early predicting septic shock through bio-data analysis based on artificial intelligence, the computing device comprising:
a processor;
a network interface;
a memory; and
a computer program loaded into the memory and executed by the processor,
wherein the computer program includes:
an instruction to collect time series-based bio-data measured in real time from a user; and
an instruction to predict an occurrence of septic shock in the user by analyzing the collected bio-data based on an artificial intelligence model, and
the instruction to predict the occurrence of the septic shock includes an instruction to analyze the bio-data at a future time point predicted based on the collected bio-data to early predict the occurrence of the septic shock at the future time point, when sepsis is detected in the user by analyzing the collected bio-data.

13. A computing device-readable recording medium, which is coupled to a computing device and on which a computer program for executing a method of early predicting septic shock through bio-data analysis based on artificial intelligence is recorded, wherein the method includes:
collecting time series-based bio-data measured in real time from a user; and
predicting an occurrence of septic shock in the user by analyzing the collected bio-data based on an artificial intelligence model, and
the predicting of the occurrence of the septic shock includes analyzing the bio-data at a future time point predicted based on the collected bio-data to early predict the occurrence of the septic shock at the future time point, when sepsis is detected in the user by analyzing the collected bio-data.
